(19) United States
(12) Patent Application Publication
(10) Pub. No.: US 2019/0079038 A1
(43) Pub. Date: Mar. 14, 2019

(54) MOISTURE SENSITIVE SHEET AND MOISTURE SENSITIVE SYSTEM

(71) Applicant: NIPPON MEKTRON, LTD., Tokyo (JP)

(72) Inventors: Hiroyasu HASEGAWA, Tokyo (JP); Akio YOSHIDA, Tokyo (JP)

(73) Assignee: NIPPON MEKTRON, LTD., Tokyo (JP)

(21) Appl. No.: 16/053,208

(22) Filed: Aug. 2, 2018

(30) Foreign Application Priority Data
Sep. 13, 2017 (JP) 2017-176154

(51) Int. Cl. G01N 27/12; H05K 1/02; A61B 5/00

(52) U.S. Cl. CPC G01N 27/121; A61B 2562/029; A61B 5/4266; H05K 1/0283

(57) ABSTRACT

A moisture sensitive sheet includes a stretchable circuit board having stretchability, a moisture absorbing film provided on a main surface of the stretchable circuit board and exhibiting an electric property changing according to an amount of moisture absorption, and a stretchable electrode provided on the main surface, having stretchability, and configured to measure the electric property of the moisture absorbing film.

### CROSS-REFERENCE TO RELATED APPLICATION

This application claims priority from Japanese Patent Application No. 2017-176154 the Japan Patent Office on Sep. 13, 2017, the entire content of which is hereby incorporated by reference.

### BACKGROUND

### 1. Technical Field

The present disclosure relates to a moisture sensitive sheet and a moisture sensitive system.

### 2. Description of the Related Art

It has been known that a sweating amount and a sweating spot are, for people, effective information for grasping the state of autonomic nerves. For example, a patient of, e.g., complications of diabetes or Graves' disease might cause a great amount of sweating. Moreover, as a symptom of a patient of heat stroke, a great amount of sweating is caused, or conversely, the amount of sweating decreases. For these reasons, a market has demand for a moisture sensitive sensor which can be used being directly attached to a person (hereinafter referred to as a “target person”) targeted for monitoring of a physical condition. Thus, a moisture sensitive sheet and a moisture sensitive system using the moisture sensitive sheet have been developed.

Considering sensitivity of the moisture sensitive sensor, the moisture sensitive sensor is, as the method for attaching the moisture sensitive sensor to the target person, preferably attached in direct contact with the body of the target person. Examples of the moisture sensitive sensor attachable to the target person in contact with the body of the target person are described in JP-A-2007-248409 and JP-A-11-101766.

A flexible humidity sensor described in JP-A-2007-248409 is a humidity sensor obtained in such a manner that an electrode is formed on a support substrate having an organic polymer plate or film shape with flexibility, hydrophobicity, and electric insulation. With this configuration, the flexible humidity sensor described in JP-A-2007-248409 can be attached to a target person along a curved shape. According to the technique described in JP-A-2007-248409, a substrate back surface of the moisture sensitive sensor having a medical tape thereon is attached to a living body.

According to a flexible moisture sensitive sensor described in JP-A-11-101766, a sensitive material mixed with soluble polymer as a polymer material is used for a sensitive portion of the sensor, the soluble polymer exhibiting responsiveness to humidity. The sensitive portion described in JP-A-11-101766 can be formed in a string shape or a fabric shape. Thus, the sensitive portion is interweaved with or sewn into a diaper or clothes, so that the sensitive portion can directly contact the skin of a living body.

### SUMMARY

A moisture sensitive sheet includes a stretchable circuit board having stretchability, a moisture absorbing film provided on a main surface of the stretchable circuit board and exhibiting an electric property changing according to an amount of moisture absorption, and a stretchable electrode provided on the main surface, having stretchability, and configured to measure the electric property of the moisture absorbing film.

### BRIEF DESCRIPTION OF THE DRAWINGS

FIG. 1 is a view for describing a moisture sensitive sheet 1 and a moisture sensitive system using the moisture sensitive sheet according to a first embodiment of the present disclosure;

FIG. 2A is an upper view of the moisture sensitive sheet according to the first embodiment, and FIG. 2B is a sectional view of the moisture sensitive sheet according to the first embodiment;

FIG. 3 is a diagram of an equivalent circuit of a stretchable electrode illustrated in FIGS. 2A and 2B;

FIG. 4 is a functional block diagram for describing an application of an information terminal device illustrated in FIG. 1;

FIG. 5 is a graph of an example of a detecting signal recorded by a logger illustrated in FIG. 4;

FIG. 6 is a view for describing another example of the moisture sensitive sheet according to the first embodiment;

FIG. 7A is an upper view of a moisture sensitive sheet according to a second embodiment, and FIG. 7B is a sectional view of the moisture sensitive sheet according to the second embodiment; and

FIG. 8A is an upper view of a moisture sensitive sheet according to a variation of the second embodiment, and FIG. 8B is a sectional view of the moisture sensitive sheet according to the variation of the second embodiment

### DESCRIPTION OF THE EMBODIMENTS

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Any of the humidity sensor described in JP-A-2007-248409 and the moisture sensitive sensor described in JP-A-11-101766 exhibits flexibility, but does not have stretchability. For this reason, when the humidity sensor described in JP-A-2007-248409 is attached to the body of the target person with the medical tape, the humidity sensor cannot follow motion of the body, and thus, a clearance is possibly formed between the humidity sensor and the body surface. Moreover, the moisture sensitive sensor described in JP-A-11-101766 is formed integrally with the clothes of the target person. Thus, when there is a clearance between the clothes and the body surface, the moisture sensitive sensor does not contact the body surface. Considering prompt detecting of sweating of the target person, there is a margin for improvement in the humidity sensor described in JP-A-2007-248409 and the moisture sensitive sensor described in JP-A-11-101766.

When a time delay from sweating of the target person to detecting of sweating is caused, detecting of, e.g., heat stroke might be delayed. Thus, a symptom of the target person possibly becomes severe.

One embodiment of the present disclosure has been made in view of the above-described points. One embodiment of the present disclosure relates to a moisture sensitive sheet and a moisture sensitive system using the moisture sensitive sheet. The moisture sensitive sheet can constantly contact a body surface of a target person to promptly detect sweating of the target person.

A moisture sensitive sheet according to the present embodiment includes a stretchable circuit board having stretchability, a moisture absorbing film provided on a main surface of the stretchable circuit board and exhibiting an electric property changing according to an amount of moisture absorption, and a stretchable electrode provided on the main surface, having stretchability, and configured to measure the electric property of the moisture absorbing film.

The moisture sensitive sheet may further include an adhesive layer formed in a partial region of the stretchable circuit board and exhibiting adhesion.

The adhesive layer may be formed such that at least a part of the moisture absorbing film is exposed on the main surface provided with the moisture absorbing film.

The stretchable circuit board may include a moisture impermeable film exhibiting low moisture permeability.

The adhesive layer may be formed at a surface on a back of the main surface provided with the moisture absorbing film such that the surface on the back of the main surface corresponding to a first region is at least partially exposed, the moisture absorbing film being formed in the first region.

The stretchable electrode may include multiple electrode portions. The adhesive layer may be formed such that the surface on the back of the main surface corresponding to a second region between the multiple electrode portions is at least partially exposed.

The stretchable circuit board may include a moisture permeable film exhibiting moisture permeability.

The moisture sensitive sheet may further include a stretchable protection film covering the moisture absorbing film and having stretchability.

A moisture sensitive system according to the present embodiment includes the moisture sensitive sheet, a property acquirer configured to acquire the electric property measured by the stretchable electrode of the moisture sensitive sheet, a calculator configured to obtain a temporal change in the electric property acquired by the property acquirer, and a signal output unit configured to output a signal based on a result of calculation of the calculator.

The present embodiment can provide the moisture sensitive sheet and the moisture sensitive system using the moisture sensitive sheet. The moisture sensitive sheet can constantly contact a body surface of a target person to promptly detect sweating of the target person

Hereinafter, embodiments of the present disclosure will be described with reference to the drawings. In all drawings, the same reference numerals are used to represent equivalent components. Further, overlapping description will be omitted as necessary. Moreover, the drawings described below are schematic views/diagrams for describing each configuration illustrated in the drawings. Thus, the drawings are not intended to limit the embodiments of the present disclosure. The schematic views/diagrams illustrate, e.g., members included in the embodiments of the present disclosure and a relationship among the members. The drawings do not necessarily illustrate, e.g., precise shapes, sizes, lengths, heights, and widths of the members included in the embodiments of the present disclosure.

### First Embodiment

<Summary>

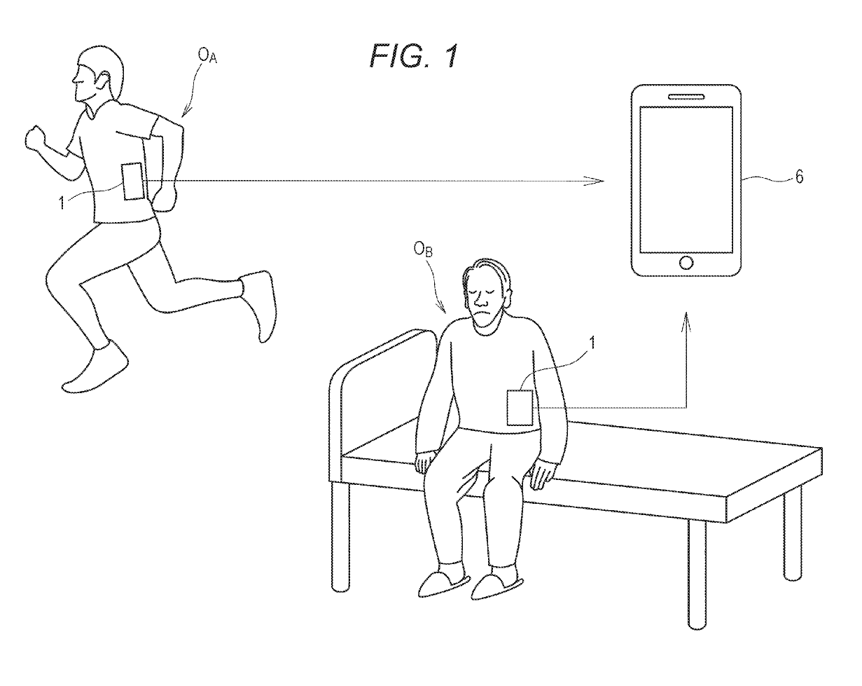

FIG. 1 is a view for describing a moisture sensitive sheet 1 and a moisture sensitive system using the moisture sensitive sheet according to a first embodiment of the present disclosure.

As illustrated in FIG. 1, the moisture sensitive system according to the first embodiment includes the moisture sensitive sheet 1 attached to, e.g., a target person O_{A} or a target person O_{B}. The moisture sensitive system according to the first embodiment can be used for prevention of heat stroke by detecting of a sweating state of a player during sports training. Alternatively, the moisture sensitive system according to the first embodiment can be used for examination of a state of an illness such as diabetes by measurement of a sweating state of a target person in a medical facility. For prevention of heatstroke, the moisture sensitive sheet 1 is directly attached to the body of the target person O_{A} during training. Moreover, for examination of the illness, the moisture sensitive sheet 1 is directly attached to the body of the target person O_{B} under examination.

The moisture sensitive sheet 1 stretches to follow motion of a body surface of the target person O_{A}, O_{B} on the body surface. Thus, even when the target person O_{A}, O_{B} moves, almost no clearance is formed between the moisture sensitive sheet 1 and the body surface. Thus, upon sweating on the body surface, the moisture sensitive sheet 1 can promptly detect sweat. With this configuration, an abnormal amount of sweating of the target person O_{A} can be promptly detected in the first embodiment. Moreover, according to the first embodiment, the timing of causing an abnormal amount of sweating of the target person O_{B} can be accurately detected. Thus, a relationship between sweating and a diet meal or exercise can be examined with high accuracy.

Note that the attachment position of the moisture sensitive sheet 1 to the target person O_{A}, O_{B} is not specifically limited. Preferably, the moisture sensitive sheet 1 is attached to a position with the highest accuracy according to the purpose of detecting. A specific attachment position includes, for example, a location assumed as having a relatively-large amount of sweating, such as the back, the side, the neck, and the thigh.

Moreover, according to the first embodiment, the moisture sensitive sheet 1 and an information terminal device 6 are connected to each other, so that a detecting result of the moisture sensitive sheet 1 can be transmitted to the information terminal device 6. When the information terminal device 6 is compact equipment being personally portable, such as a smartphone, the target person O_{A}, O_{B} can wear the information terminal device 6 together with the moisture sensitive sheet 1. With this configuration, the moisture sensitive sheet 1 and the information terminal device 6 can be connected to each other via a signal line. In the first embodiment, the moisture sensitive sheet 1 may be provided with a compact transmitter to communicate with the information terminal device 6 wirelessly.

With this configuration, the information terminal device 6 can receive a detecting signal of the moisture sensitive sheet 1 to emit sound and/or light. By the sound and/or the light emitted from the information terminal device 6, the target person O_{A}, O_{B} can recognize an abnormality in physical conditions of themselves. Thus, the target person O_{A} during training can promptly move on to treatment of an illness. Further, the target person O_{B} can notify an abnormality to, e.g., a person in charge of examination to receive an instruction.

Hereinafter, each configuration for implementing the above-described system will be described.

<Moisture Sensitive Sheet>

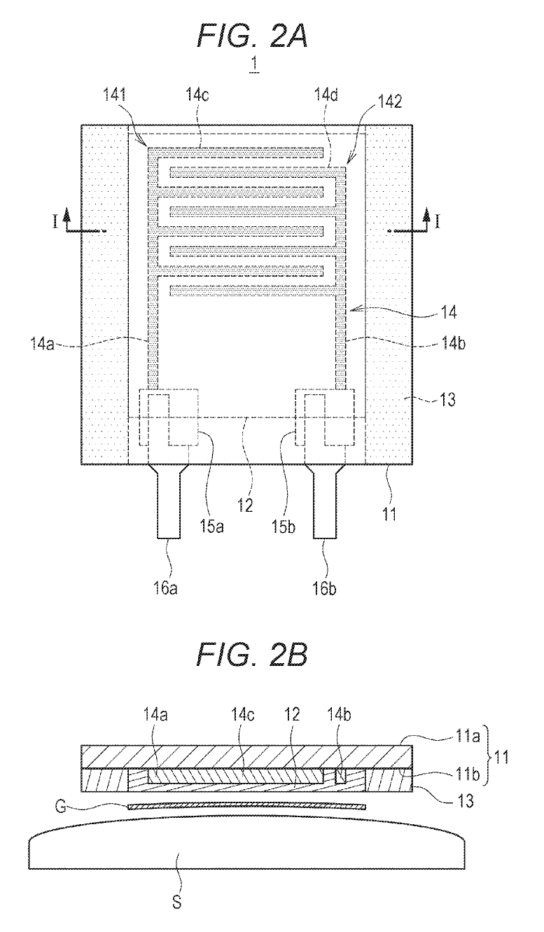

FIGS. 2A and 2B are views for describing the moisture sensitive sheet 1 of the first embodiment. FIG. 2A is an outer view (hereinafter referred to as an “upper view”) of the moisture sensitive sheet 1 attached to a living body S. FIG. 2B is a view of a section, viewed from a direction of an arrow I-I illustrated in FIG. 2A, of the moisture sensitive sheet 1 cut along the arrow I-I.

The moisture sensitive sheet 1 includes a stretchable circuit board 11 having stretchability, a moisture absorbing film 12, and a stretchable electrode 14. The moisture absorbing film 12 is provided on a main surface 11b of the stretchable circuit board 11, and exhibits electric properties changing according to the amount of moisture absorption. The stretchable electrode 14 is provided on the main surface 11b, and have stretchability. Moreover, the stretchable electrode 14 is configured to measure the electric properties of the moisture absorbing film 12.

The moisture sensitive sheet 1 further has an adhesive layer 13 formed in a partial region of the stretchable circuit board 11 and exhibiting adhesion. In the first embodiment, such a moisture sensitive sheet 1 is attached to the body (hereinafter referred to as a “living body”) S of the target person O_{A}, O_{B} with gauze G interposed therebetween.

Hereinafter, the above-described configurations will be sequentially described.

(Stretchable Circuit Board)

The stretchable circuit board 11 is a sheet-shaped member configured to stretch in at least one of in-plane directions. The stretchable circuit board 11 may preferably stretch in two of the in-plane directions. The stretchability of the stretchable circuit board 11 in the in-plane directions may exhibit isotropy or anisotropy. When the stretchability of the stretchable circuit board 11 in the in-plane directions exhibits the anisotropy, the stretchability varies according to multiple directions in the plane of the stretchable circuit board 11. Preferably, a material contained in the stretchable circuit board 11 can include, but not limited to, elastomer materials such as nitrile rubber, latex rubber, urethane-based elastomer, and silicone-based elastomer. Specifically, even when the moisture sensitive sheet 1 is attached to a human body skin with a urethane-based elastomer sheet for medical use, high safety can be obtained. The “sheet shape” described herein indicates a shape having a sufficiently-larger area as compared to a thickness. The thickness or area of the sheet is not specifically defined. The stretchable circuit board 11 may be formed of a single layer including a single sheet-shaped member. Alternatively, the stretchable circuit board 11 may be formed of multiple layers including multiple sheet-shaped stretchable circuit boards.

Moreover, the “main surface” described in the first embodiment indicates a surface obviously having a larger area than other surfaces (surfaces in a width direction) of the sheet-shaped member. The stretchable circuit board 11 has a main surface 11a and a main surface 11b.

The thickness of the stretchable circuit board 11 is not specifically limited. Note that the thickness of the stretchable circuit board 11 is preferably equal to or less than 100 μm, from a viewpoint that stretching or motion of a target object (a target surface such as a living body surface) to which the stretchable circuit board 11 is attached is not inhibited. The thickness of the stretchable circuit board 11 is more preferably equal to or less than 25 μm, and much more preferably equal to or less than 10 μm.

As described above, according to the first embodiment, use of the thin sheet-shaped stretchable circuit board 11 having the stretchability does not inhibit motion of the target person O_{A}, O_{B} to which the moisture sensitive sheet 1 is attached. Thus, an uncomfortable feeling due to attachment can be sufficiently reduced. Moreover, the elastomer material exhibits moisture permeability and air permeability. Thus, the uncomfortable feeling on the target person O_{A}, O_{B} when the moisture sensitive sheet 1 is attached to the body surface can be further reduced.

(Moisture Absorbing Film)

The moisture absorbing film 12 is a film exhibiting the electric properties changing according to the amount of moisture absorption. In the moisture absorbing film 12 according to the first embodiment, an impedance changes as the electric properties. Note that the moisture absorbing film 12 is not limited to the configuration in which the impedance changes. A current flowing in the moisture absorbing film 12 may change. Alternatively, a voltage value in the case of supplying direct current to the moisture absorbing film 12 may change. Note that in the first embodiment, the moisture absorbing film 12 covers the entirety of the stretchable electrode 14. However, the first embodiment is not limited to such a configuration. The moisture absorbing film 12 may cover at least a part of the stretchable electrode 14.

In the first embodiment, the moisture absorbing film 12 is an ion conductive film. In the ion conductive film, a greater amount of water absorption results in higher conductivity. Thus, conduction easily occurs between a first electrode portion 141 and a second electrode portion 142 included in the stretchable electrode 14.

The moisture absorbing film 12 may be a PVA-silica composite body in which polymer is dispersed, the polymer exhibiting responsiveness to the amount of water absorption. Such a moisture absorbing film 12 can be formed by the following method, for example. The stretchable circuit board 11 is dipped in a solution mixed with the polymer, the stretchable electrode 14 having been patterned on the stretchable circuit board 11. Thereafter, the stretchable circuit board 11 is slowly lifted at constant speed, and then, is dried. The solution for use in dipping the stretchable circuit board 11 includes, for example, a solution obtained in such a manner that tetraethoxysilane (TEOS) and hydrochloric acid are added to polyvinyl alcohol (PVA) and sodium polystyrene sulfonate (PSSNa) dissolved in reflux using a solvent mixture (4:1) of water and ethanol. The hydrochloric acid is herein used as a catalyst. The method for drying the stretchable circuit board 11 may be natural drying or artificial drying. At this point, close attention is necessary to avoid application of vibration to the stretchable circuit board 11.

Alternatively, the moisture absorbing film 12 can be also formed by the following method. A liquid mixture is applied to the stretchable electrode 14, the liquid mixture having been obtained by stirrer mixing of 99 mass % of a moisture sensitive solution and 1 mass % of styrene butadiene latex emulsion. The moisture sensitive solution described herein is obtained in such a manner that acrylic resin having a sulfonic acid group at a terminal thereof is dissolved in 50 mass % of pure water and 50 mass % of methanol. Application to the stretchable electrode 14 can be performed in such a manner that, e.g., the liquid mixture filling a discharger is applied to the stretchable electrode 14, and is dried for one hour at 100° C.

(Stretchable Electrode)

The stretchable electrode 14 includes the first electrode portion 141 and the second electrode portion 142. The first electrode portion 141 and the second electrode portion 142 are paired with each other. The first electrode portion 141 includes a first longitudinal electrode portion 14a and first lateral electrode portions 14c. The second electrode portion 142 includes a second longitudinal electrode portion 14b and second lateral electrode portions 14d. The stretchable electrode 14 is a so-called comb electrode. The first electrode portion 141 has the first longitudinal electrode portion 14a and the multiple first lateral electrode portions 14c. Each of the multiple first lateral electrode portions 14c is connected to the first longitudinal electrode portion 14a while extending toward a second longitudinal electrode portion 14b.

Moreover, the second electrode portion 142 has the second longitudinal electrode portion 14b and the multiple second lateral electrode portions 14d. Each of the multiple second lateral electrode portions 14d is connected to the second longitudinal electrode portion 14b while extending toward the first longitudinal electrode portion 14a. The first lateral electrode portions 14c and the second lateral electrode portions 14d are formed at equal pitch, and are alternately arranged. The first lateral electrode portions 14c and the second lateral electrode portions 14d are preferably arranged at such a pitch that an electrode width to an electrode interval is about 1:1 to 1:2, for example. In the stretchable electrode 14 as the comb electrode, a smaller interval between the first lateral electrode portion 14c and the second lateral electrode portion 14d results in more conduction between the first electrode portion 141 and the second electrode portion 142.

As illustrated in FIG. 2A, the first electrode portion 141 is electrically connected to an external electrode 15a and a wiring portion 16a. The second electrode portion 142 is electrically connected to an external electrode 15b and a wiring portion 16b. Preferably, the external electrodes 15a, 15b are formed of a stretchable conductive material at the same time as formation of the stretchable electrode 14. In this manner, flexibility of the moisture sensitive sheet 1 is not impaired even after the external electrodes 15a, 15b have been attached to the stretchable electrode 14 in the first embodiment. Moreover, a space between the external electrode 15a, 15b and the main surface 11b may be filled with a fixing enhancement adhesive such as a conductive highly-flexible adhesive, and in this manner, the external electrodes 15a, 15b formed of a conductive flexible material may be physically fixed to the stretchable electrode 14. Even in such formation of the external electrodes 15a, 15b, the flexibility of the moisture sensitive sheet 1 is not impaired. The external electrode 15a and the wiring portion 16a are preferably integrated with each other. Further, the external electrode 15b and the wiring portion 16b are also preferably integrated with each other.

The external electrode 15a connects the first electrode portion 141 and external equipment. The external electrode 15b connects the second electrode portion 142 and the external equipment. In the first embodiment, the external electrodes 15a, 15b are connected to a not-shown AC power supply. Thus, AC power is supplied to the first electrode portion 141 and the second electrode portion 142.

At this point, in the first embodiment, a resistance value between the first electrode portion 141 and the second electrode portion 142 is measured by an AC impedance method. The impedance obtained by diverging of a high-frequency pulse is taken as a value substantially equal to the resistance value.

The stretchable electrode 14 contains a conductive material. Thus, the stretchable electrode 14 exhibits conductivity. A material with favorable conductivity can be selected as the conductive material. The material with favorable conductivity includes, for example, silver, gold, platinum, carbon, copper, aluminum, cobalt, nickel, and alloy thereof. The shape of the conductive material is not specifically limited, but may be a particle form such as granulated powder or powder. The particle form is not specifically limited, but may be, e.g., a spherical shape, a needle shape, a flake shape, a nanowire shape or the like. A particle aspect ratio may fall within a range of equal to or greater than 1 and equal to or less than 100, and specifically a range of equal to or greater than 1 and equal to or less than 50. The aspect ratio described herein means the ratio between the longest dimension and the shortest dimension of a three-dimensional body. When the aspect ratio of the particle contained in the stretchable electrode 14 falls within a range of equal to or greater than 5 and equal to or less than 20, a change in resistance when the stretchable circuit board 11 stretching in the in-plane directions deforms in a length direction can be reduced.

The stretchable electrode 14 preferably further contains a resin binder. That is, the stretchable electrode 14 according to the first embodiment is formed with the conductive material. This conductive material is obtained in such a manner that conductive particles are dispersed in a resin material while conductive resin is mixed with the resin material. Since the stretchable electrode 14 contains the resin binder, rupturing of the stretchable electrode 14 due to stretching is reduced. The resin binder includes, but not limited to, a binder containing, as a main component, resin such as urethane or polyester, and a thermoplastic elastomer material such as silicone rubber, for example. Preferably, a resin binder with a low Young's modulus is selected such that the stretchable electrode 14 in the form of a coating has an elastic modulus equal to or less than that of the stretchable circuit board 11. A single type of elastomer material may be used. Alternatively, a mixture of multiple types of elastomer materials may be used.

The method for manufacturing the stretchable electrode 14 is not specifically limited. In the first embodiment, the stretchable electrode 14 is formed by a printing method. That is, the stretchable electrode 14 is a printed pattern formed in such a manner that conductive paste having stretchability is printed and applied onto the main surface 11b. The printing method is not specifically limited. The printing method may include, a screen printing method, an inkjet printing method, a gravure printing method, an offset printing method and the like. Of these methods, screen printing is preferably used, considering fine resolution properties and film thickness stability. In the case of forming the stretchable electrode 14 by the printing method, conductive paste prepared to contain the above-described conductive particles, the above-described resin binder, and an organic solvent is preferably used. Stretchable conductive paste containing, as a main component, metal particles such as silver is used for the stretchable electrode 14, so that a stretch rate falling within a range of equal to or higher than about 50% and equal to or lower than about 70% can be realized, for example. Thus, a wiring line with excellent stretching properties can be formed.

The thickness and width dimensions of the stretchable electrode 14 can be determined based not only on resistivity under no load and a change in resistance upon stretching of the stretchable circuit board 11, but also on limitations of the thickness and width dimensions of the entirety of the stretchable circuit board 11. The width dimensions of the stretchable electrode 14 are preferably equal to or less than 1000 μm, more preferably equal to or less than 500 μm, and much more preferably equal to or less than 200 μm. The above-described width dimensions of the stretchable electrode 14 are based on the point of view that the stretchable electrode 14 follows a change in the dimensions upon stretching of the stretchable circuit board 11 while favorable stretchability is ensured. The thickness dimensions of the stretchable electrode 14 may be equal to or less than 25 μm, and preferably falls within a range of equal to or greater than 10 μm and equal to or less than 15 μm.

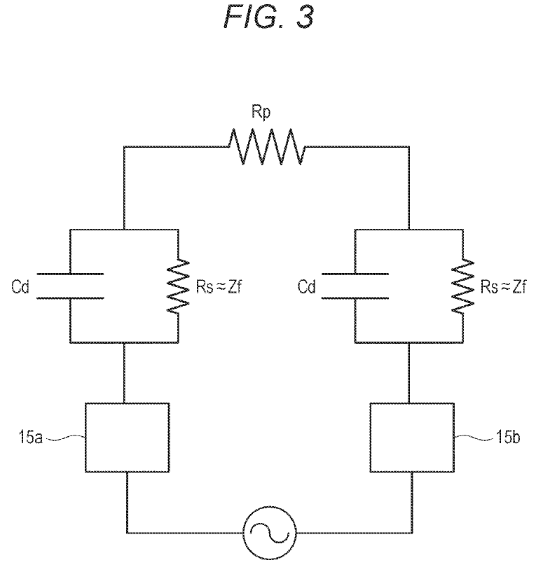

FIG. 3 is a diagram of an equivalent circuit of the stretchable electrode 14 illustrated in FIGS. 2A and 2B. In the first embodiment, when a voltage between the external electrode 15a and the external electrode 15b in FIG. 3 is measured, the equivalent circuit illustrated in FIG. 3 can be analyzed by the AC impedance method. The impedance Zp of the moisture sensitive sheet 1 derived from the equivalent circuit illustrated in FIG. 3 is represented by Expression (1) below. Note that signs in Expression (1) are as follows.

Zp=Rp+2Rs/(1+Rs⋅iwCd) Expression (1)

Rp: Solution Resistance, Rs: Polarization Resistance, Cd: Double Layer Capacitance

Note that in the first embodiment, the value of the polarization resistance Rs in Expression (1) is taken as a value substantially equal to the value of a faradaic impedance Zf. The faradaic impedance Zf is a value resulting from movement of charge and substance on an electrode interface.

When an infinite high frequency pulse, actually a high frequency pulse of equal to or higher than 10 KHz, is applied to the equivalent circuit illustrated in FIG. 3, iwCd of Expression (1) goes to infinity (diverges). As a result, Expression (1) is represented as in Expression (2) below.

Zp≈Rp Expression (2)

Expression (2) shows that the impedance Zp is a value substantially equal to the solution resistance Rp. The solution resistance Rp is the resistance value of the moisture absorbing film 12. The resistance value obtained as described above is herein taken as the resistance value R of the moisture absorbing film 12. The resistance value R linearly changes according to the degree of moisture absorption of the moisture absorbing film 12. Thus, a more preferable value is obtained in the case of taking the measured resistance value R as the solution resistance Rp than in the case of taking the impedance Zp as the solution resistance Rp.

The resistance value when direct current is applied to the stretchable electrode 14 from a DC power supply is obtained in such a manner that the polarization resistance Rs and the solution resistance Rp are added up. The case of taking this resistance value as the resistance value R of the moisture absorbing film 12 lacks appropriateness. For this reason, the resistance value R of the moisture absorbing film 12 is preferably measured by the above-described AC impedance method.

(Adhesive Layer)

As illustrated in FIG. 2B, the adhesive layer 13 is, on the main surface 11b on which the moisture absorbing film 12 is provided, formed such that the moisture absorbing film 12 is at least partially exposed. This feature is provided because the moisture sensitive sheet 1 is attached while the surface of the moisture sensitive sheet 1 on the side provided with the moisture absorbing film 12 faces the living body S. That is, the moisture sensitive sheet 1 according to the first embodiment is configured such that the moisture absorbing film 12 faces the living body S via the gauze G. Sweat generated from the living body S flows toward the moisture absorbing film 12 through the gauze G. Thus, when the adhesive layer 13 covers the entire area of the moisture absorbing film 12, the moisture absorbing film 12 cannot sufficiently absorb moisture. As a result, the moisture sensitive sheet 1 cannot fulfill the function of detecting humidity.

The thickness of the adhesive layer 13 may be equal to that of the moisture absorbing film 12. Alternatively, the adhesive layer 13 may be thicker or thinner than the moisture absorbing film 12. When the adhesive layer 13 is thicker or thinner than the moisture absorbing film 12, the moisture sensitive sheet 1 is attached to the living body S while an end portion of the stretchable circuit board 11 is slightly stretching or contracting, the adhesive layer 13 being applied to the stretchable circuit board 11.

For at least a part of the moisture absorbing film 12, the percentage of the exposed portion of the moisture absorbing film 12 is not specifically defined. As long as an area where the resistance value measured by the external electrode 15a and the external electrode 15b changes according to the sweat generated from the living body S can be ensured, any percentage may be employed as the percentage of the exposed portion of the moisture absorbing film 12.

In the moisture sensitive sheet 1 of the first embodiment including the configuration in which the moisture absorbing film 12 faces the living body S, the stretchable circuit board 11 may include a moisture impermeable film exhibiting low moisture permeability. The moisture impermeable film described herein means a film exhibiting significantly low moisture permeability. The moisture permeability means the amount of water vapor passing through a membranous substance per unit area within a certain time. Under standards for moisture permeability, air on one side is, using the membranous substance as a boundary surface, held at a relative humidity of 90% at a temperature of 25° C. or 40° C. while air on the other side is held in a dry state by a moisture absorbent material. The moisture permeability is represented by a value obtained in such a manner that the mass (g) of water vapor passing through the boundary surface within 24 hours in the above-described state is converted into a value per square meter of the membranous substance. The moisture impermeable film according to the first embodiment indicates, for example, a film having a moisture permeability of equal to or less than 100 g.

When the stretchable circuit board 11 includes the moisture impermeable film, moisture of sweat absorbed by the moisture absorbing film 12 stays in the moisture absorbing film 12 without passing through the stretchable circuit board 11. Thus, in the moisture absorbing film 12, the absorbed moisture sufficiently spreads. Consequently, a signal output from the moisture sensitive sheet 1 can be stabilized. Note that the first embodiment is not limited to the configuration in which the stretchable circuit board 11 includes the moisture impermeable film. The stretchable circuit board 11 may include a moisture permeable film exhibiting high moisture permeability. With this configuration, the moisture of the sweat absorbed by the moisture absorbing film 12 passes upward through the stretchable circuit board 11. Thus, the moisture sensitive sheet 1 reacts, with high accuracy, to a change in the amount of sweat generated from the living body S. As a result, an actual sweating amount can be accurately measured.

<Moisture Sensitive System>

Next, the moisture sensitive system of the first embodiment utilizing the above-described moisture sensitive sheet 1 will be described.

As illustrated in FIG. 1, the moisture sensitive system of the first embodiment includes the moisture sensitive sheet 1 attached to the body of the target person O_{A}, O_{B} and the information terminal device 6 illustrated in FIG. 1. In the first embodiment, the information terminal device 6 includes an application for moisture detecting.

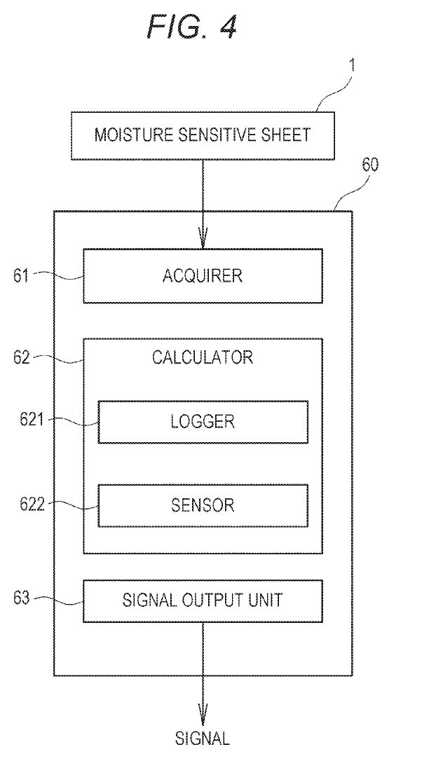

FIG. 4 is a functional block diagram for describing a moisture sensitive controller 60 of the information terminal device 6. The moisture sensitive controller 60 has an acquirer 61 and a calculator 62. The acquirer 61 is a property acquirer configured to acquire the electric properties measured by the stretchable electrode 14 of the moisture sensitive sheet 1. The calculator 62 is configured to obtain a temporal change in the resistance value obtained by the acquirer 61. Since the calculator 62 obtains the temporal change in the resistance value, the calculator 62 has a logger 621 and a sensor 622, the logger 621 being configured to record the resistance value in chronological order, the sensor 622 being configured to detect, from the record in the logger 621, a resistance value changing time and a resistance value changing degree. Moreover, the moisture sensitive controller 60 has a signal output unit 63 configured to output a signal based on a result of calculation of the calculator 62.

In the first embodiment in which the information terminal device 6 is used to process the detecting signal of the moisture sensitive sheet 1 to detect abnormal sweating, e.g., a sound function, a music reproduction function, or a vibration function of the information terminal device 6 may be utilized as the signal output unit 63. The moisture sensitive controller 60 illustrated in FIG. 4 is software (an application program) operable on the information terminal device 6, and is operable using existing hardware of the information terminal device 6.

(Operation)

The moisture sensitive controller 60 illustrated in FIG. 4 operates as follows. That is, the acquirer 61 acquires, with or without a wire, the detecting signal output from the moisture sensitive sheet 1. The acquirer 61 is implemented by an input interface or a receiving function for the detecting signal. The detecting signal acquired by the acquirer 61 is passed on to the calculator 62. In the calculator 62, the detecting signal is recorded in input order by the logger 621.

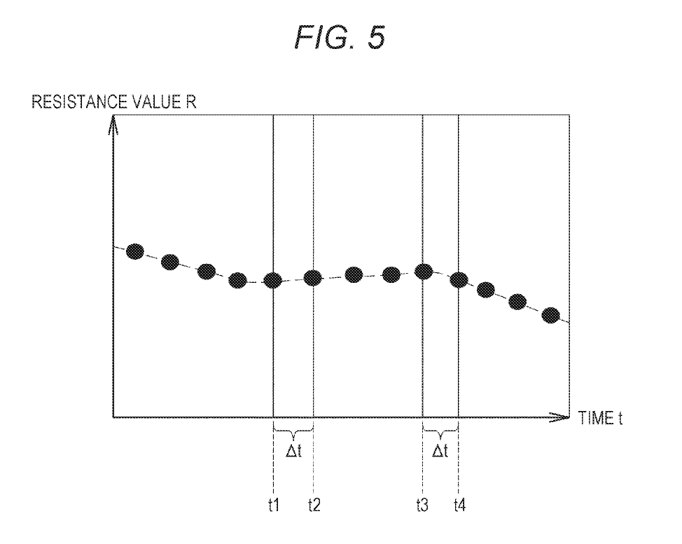

FIG. 5 is a graph of an example of the detecting signal recorded by the logger 621. In the graph of FIG. 5, the horizontal axis represents a time t, and the vertical axis represents the resistance value R indicated by the detecting signal. FIG. 5 illustrates the example of recording of the detecting signal of the moisture sensitive sheet 1 attached to the target person O_{B}.

The moisture sensitive sheet 1 outputs, as the detecting signal, the resistance value R between the first electrode portion 141 and the second electrode portion 142 in every time Δt. The acquirer 61 sequentially acquires the detecting signal. The logger 621 records, in chronological order, the acquired resistance value in association with the time. The sensor 622 detects, from the record in the logger 621, a time differential value (hereinafter referred to as “ΔR/Δt”) of the resistance value. ΔR/Δt continuously shows a negative value after the start of measurement until right before a time point t1 while ΔR/Δt shows a positive value between the time point t1 and a time point t2. Such a phenomenon occurs due to the following reason. The resistance value of the moisture absorbing film 12 is gradually stabilized right after the moisture sensitive sheet 1 has been attached to the living body S. Thereafter, the stretchable electrode 14 stretches, or moisture is vaporized from the moisture absorbing film 12.

Further, ΔR/Δt shows the positive value until a time point t3 while ΔR/Δt shows the negative value between the time point t3 and a time point t4. Such a phenomenon occurs due to a decrease in the resistance value caused by absorption of sweat from the living body S, the absorption performed by the moisture absorbing film 12. Note that when ΔR/Δt is 0, it is assumed that sweating, moisture vaporization from the moisture absorbing film 12, and stretching of the stretchable electrode 14 are in a steady state.

In the first embodiment, when the value of ΔR/Δt (or an absolute value of negative ΔR/Δt) reaches equal to or greater than a preset threshold, the sensor 622 may detect abnormal sweating to instruct the signal output unit 63 to output the signal. Alternatively, in the first embodiment, when a time or the number of times that the value of ΔR/Δt continuously shows the negative value reaches a preset threshold, the sensor 622 may instruct the signal output unit 63 to output the signal. The threshold for the value (the absolute value) of ΔR/Δt and the threshold for the number of times can be determined according to the purpose for observing sweating, for example.

For example, in the case of determining the presence or absence of complications of diabetes, the sensor 622 can be set such that detecting of the absolute value, which is equal to or greater than the threshold, of negative ΔR/Δt is detected within a certain time range after a lapse of a set time after a meal.

Moreover, in the case of monitoring sweating for prevention of heat stroke, the sensor 622 can be set such that, e.g., the negative value of ΔR/Δt continuously detected the number of times equal to or greater than the threshold is detected. Further, in the case of detecting the onset of heat stroke, it may be configured such that when the positive value of ΔR/Δt or a ΔR/Δt of zero is detected after the negative value of ΔR/Δt has been continuously detected, the signal output unit 63 outputs a warning indicating the probability of the onset of heat stroke. Such processing is performed based on the following symptoms. One cannot stop sweating as an initial symptom of heat stroke, and on the other hand, does not sweat in a moderate level of heat stroke.

The moisture sensitive sheet 1 according to the first embodiment as described above is used such that the stretchable circuit board 11 having the stretchability is attached to the target person O_{A}, O_{B}. Thus, the moisture sensitive sheet 1 stretches to follow motion of the target person O_{A}, O_{B} while constantly contacting the body surface of the target person O_{A}, O_{B}. Thus, almost no clearance is formed between the moisture sensitive sheet 1 and the target person O_{A}, O_{B}. Consequently, sweating of the target person O_{A}, O_{B} can be promptly detected. Moreover, in the moisture sensitive sheet 1 according to the first embodiment, the stretchable circuit board 11 includes the moisture impermeable film, and therefore, the sweat generated from the surface of the living body S sufficiently extends across the moisture absorbing film 12. Thus, a stable resistance value can be obtained.

Further, when the moisture sensitive system is built using the above-described moisture sensitive sheet 1, the state of illness and a body condition due to autonomic nerve disorder such as heat stroke can be evaluated in an objective way.

(Variation)

Note that the moisture sensitive system of the first embodiment is not limited to the above-described configuration. For example, according to the moisture sensitive system described above, the resistance value between the first electrode portion 141 and the second electrode portion 142 is output from the moisture sensitive sheet 1, and the information terminal device 6 performs processing based on this resistance value. However, in the moisture sensitive system of the first embodiment, at least some of the functions of the moisture sensitive controller 60 illustrated in FIG. 4 may be provided at the moisture sensitive sheet 1.

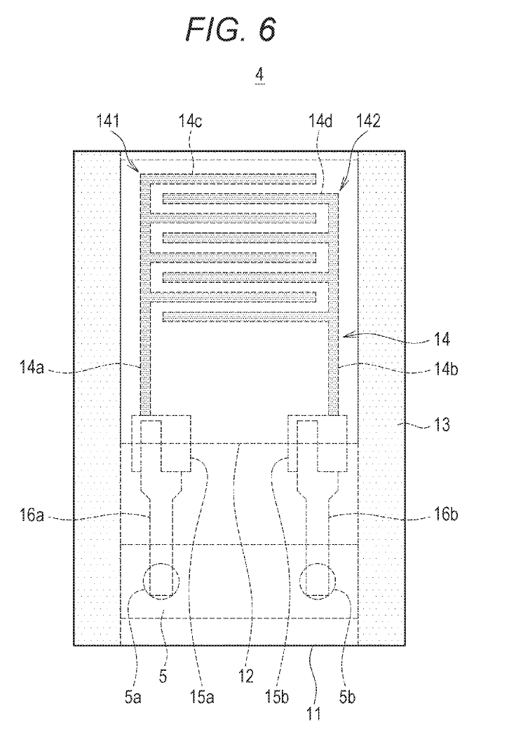

FIG. 6 is a view for describing a moisture sensitive sheet 4 (a variation) applied to the moisture sensitive system of the first embodiment including the above-described configuration. The moisture sensitive sheet 4 illustrated in FIG. 6 is different from the moisture sensitive sheet 1 illustrated in FIG. 2 in that a control circuit 5 is provided. The control circuit 5 may include the acquirer 61 and the calculator 62 of the functions illustrated in FIG. 4, for example. The control circuit 5 may output, with or without a wire, the detecting result of the sensor 622 to the information terminal device 6, thereby generating, e.g., sound and/or vibration at the information terminal device 6. Moreover, in this configuration, the moisture sensitive controller 60 is not necessarily the application program operable in a versatile information terminal device, and may be a dedicated system such as an embedded processor. The control circuit 5 can be configured as a compact integrated circuit (IC) such as a one-chip microcomputer.

The control circuit 5 includes a terminal 5a and a terminal 5b. The terminal 5a and the terminal 5b are paired with each other. The wiring portion 16a and the wiring portion 16b overlap with the terminal 5a and the terminal 5b, respectively. FIG. 6 illustrates an example where the control circuit 5 is mounted on the stretchable circuit board 11, but the present variation is not limited to such a configuration. For example, the control circuit 5 may be provided on a substrate having stretchability, the substrate being provided separately from the stretchable circuit board 11. Alternatively, the control circuit 5 may be mounted on the stretchable circuit board 11 on which a film having a thickness in such an extent that the stretchability of the moisture sensitive sheet 1 is not impaired and being harder than the stretchable circuit board 11 is provided.

### Second Embodiment

Next, a second embodiment of the present disclosure will be described. A moisture sensitive sheet 2 according to a second embodiment is different from the moisture sensitive sheet 1 according to the first embodiment in that a stretchable electrode 14 and a moisture absorbing film 12 are formed outward while the moisture sensitive sheet 2 is attached to a living body S.

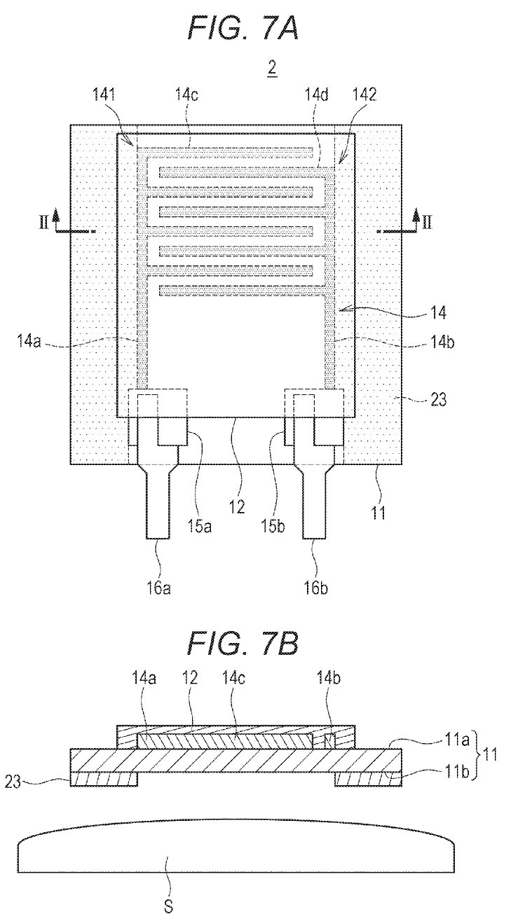

FIGS. 7A and 7B are views for describing the moisture sensitive sheet 2 according to the second embodiment. FIG. 7A is an upper view of the moisture sensitive sheet 2, and FIG. 7B is a view of a section, viewed from a direction of an arrow II-II illustrated in FIG. 7A, of the moisture sensitive sheet 2 cut along the arrow II-II. As illustrated in FIG. 7B, in the moisture sensitive sheet 2 according to the second embodiment, the stretchable electrode 14 and the moisture absorbing film 12 are formed on a main surface 11a of a stretchable circuit board 11.

In the moisture sensitive sheet 2 according to the second embodiment described above, the stretchable electrode 14 and the moisture absorbing film 12 are formed on a surface on the back of the surface of the moisture sensitive sheet 2 attached to the living body S. Thus, in the moisture sensitive sheet 2, moisture of sweat generated from the living body S is absorbed by the moisture absorbing film 12 through a main surface 11b of the stretchable circuit board 11. Thus, the stretchable circuit board 11 of the moisture sensitive sheet 2 is a moisture permeable film exhibiting moisture permeability. Note that moisture permeability of the moisture permeable film in the second embodiment indicates, for example, a moisture permeability of equal to or greater than 2000 g.

In the moisture sensitive sheet 2 with the moisture absorbing film 12 formed on the main surface 11a, when the entire area of the surface (the main surface 11b) on the back of the region where the moisture absorbing film 12 is formed on the main surface 11a is covered with an adhesive layer 23, moisture cannot penetrate from the main surface 11b to the moisture absorbing film 12. As a result, the moisture absorbing film 12 cannot fulfill a moisture detecting function. For this reason, the adhesive layer 23 of the second embodiment is formed on the surface (the main surface 11b) on the back of the main surface 11a provided with the moisture absorbing film 12 such that the surface on the back of the main surface 11a corresponding to a region (a first region) is at least partially exposed, the moisture absorbing film 12 being formed in the region (the first region).

For at least a part of the surface on the back of the region of the main surface 11a, the percentage of the exposed portion in the back surface is not specifically defined, the moisture absorbing film 12 being formed in the region. As long as an area where a resistance value measured by an external electrode 15a and an external electrode 15b changes according to the sweat generated from the living body S can be ensured, any percentage may be employed as the percentage of the exposed portion in the back surface. That is, the surface, of the main surface 11b, on the back of the region of the main surface 11a includes at least a portion where no adhesive layer 23 is formed, the moisture absorbing film 12 being formed in the region.

Further, in the moisture sensitive sheet 2 configured such that the moisture of the sweat is absorbed by the moisture absorbing film 12 through the stretchable circuit board 11, the moisture absorbing film 12 cannot absorb the sweat from the position of a first electrode portion 141 nor a second electrode portion 142. In the second embodiment, the stretchable electrode 14 includes multiple electrode portions, i.e., the first electrode portion 141 and the second electrode portion 142. Thus, in the second embodiment, the sweat is absorbed by the moisture absorbing film 12 through a clearance between the first electrode portion 141 and the second electrode portion 142 and/or a clearance between a first lateral electrode portion 14c and a second lateral electrode portion 14d. In the second embodiment with such a mechanism, when the back surface of the stretchable circuit board 11 corresponding to the clearances generated in the stretchable electrode 14 is covered with the adhesive layer 23, the sweat is not absorbed by the moisture absorbing film 12, and for this reason, the moisture absorbing film 12 cannot fulfill the moisture detecting function.

For the above-described reasons, the adhesive layer 23 of the second embodiment is formed such that the surface on the back of the main surface 11a corresponding to a region (a second region) between the first electrode portion 141 and the second electrode portion 142 is at least partially exposed.

According to the above-described second embodiment, the moisture of the sweat is absorbed concentrating on a space between the first electrode portion 141 and the second electrode portion 142. With this configuration, the resistance between the first electrode portion 141 and the second electrode portion 142 changes, with high accuracy, according to the amount of moisture absorption.

(Variation)

The second embodiment is not limited to the above-described moisture sensitive sheet 2. For example, the moisture absorbing film 12 is exposed on an outermost surface when the moisture sensitive sheet 2 is attached to a body surface of a target person O_{A}, O_{B}. In such a moisture sensitive sheet 2, e.g., moisture contained in external air is absorbed by the moisture absorbing film 12, or moisture absorbed by the moisture absorbing film 12 is evaporated and released to the outside. This might influence detecting of a sweating amount. Moreover, the moisture absorbing film 12 is exposed without protection, and for this reason, the moisture absorbing film 12 might be damaged, for example, when the moisture absorbing film 12 is contacted.

In view of the above-described points, the moisture sensitive sheet of the second embodiment may further have a stretchable protection film 18 covering the moisture absorbing film 12 and having stretchability.

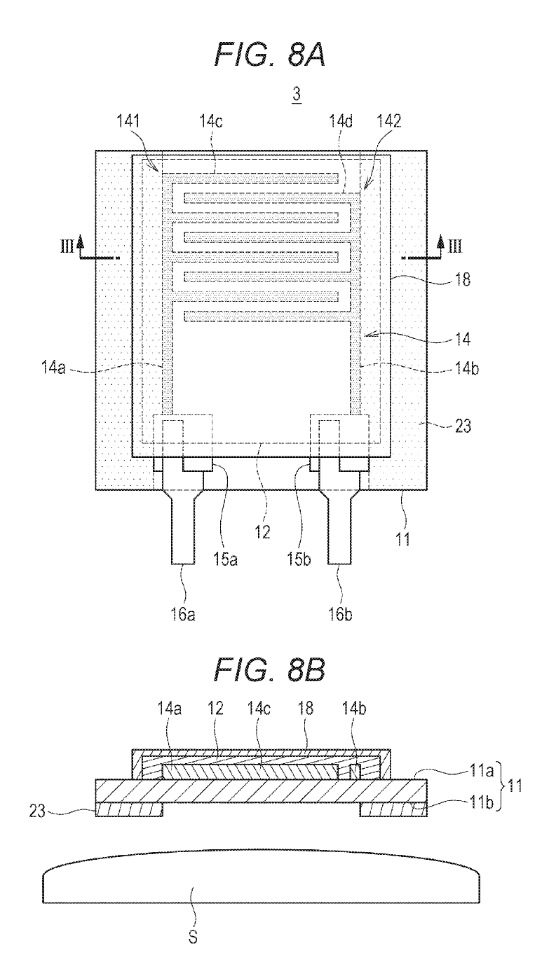

FIGS. 8A and 8B are views of a moisture sensitive sheet 3 of the variation of the second embodiment. FIG. 8A is an upper view of the moisture sensitive sheet 3, and FIG. 8B is a view of a section, viewed from a direction of an arrow III-III illustrated in FIG. 8A, of the moisture sensitive sheet 3 cut along the arrow III-III. The stretchable protection film 18 covers an entire area of the moisture absorbing film 12 to prevent or reduce the influence of the external air on the moisture absorbing film 12 and contact to the moisture absorbing film 12.

Preferably, the stretchable protection film 18 is a film having stretchability and air permeability, but exhibiting substantially no moisture permeability. A material used for the stretchable protection film 18 can include, for example, an elastomer material. Alternatively, in the stretchable protection film 18, the common resin material to that of the stretchable circuit board 11 may be used. With this configuration, the moisture absorbing film 12 can be protected without impairing of stretchability of the stretchable circuit board 11. The stretchable protection film 18 can be produced in such a manner that elastomer-based paste is applied onto the moisture absorbing film 12 and is dried. As other alternatives, the stretchable protection film 18 produced in a sheet shape in advance may be attached to the moisture absorbing film 12, or may be bonded to the moisture absorbing film 12 with an adhesive.

The thickness of the stretchable protection film 18 is not specifically limited. From the point of view that the stretchability of the stretchable circuit board 11 is not inhibited, the thickness of the stretchable protection film 18 is preferably equal to or less than 100 μm, more preferably equal to or less than 50 μm, and much more preferably equal to or less than 30 μm.

According to the above-described second embodiment, the moisture of the sweat reaches the moisture absorbing film 12 after having been diffused in the stretchable circuit board 11 exhibiting air permeability. Moreover, the moisture is absorbed by the moisture absorbing film 12 through the space between the first electrode portion 141 and the second electrode portion 142 of the stretchable electrode 14. Thus, the resistance value between the first electrode portion 141 and the second electrode portion 142 exhibits high responsiveness to moisture absorption. Consequently, the moisture sensitive sheet 2 according to the second embodiment can exhibit high sensitivity to a change in the sweating amount. Further, according to the variation, absorption of the moisture from the external air by the moisture absorbing film 12 and contact to the moisture absorbing film 12 are prevented or reduced. With this configuration, the sweating amount can be more accurately detected. Further, the moisture sensitive sheet 3 with scratch resistance can be provided.

The above-described embodiments and examples include the following technical ideas.

A moisture sensitive sheet <1> includes a stretchable circuit board having stretchability, a moisture absorbing film provided on a main surface of the stretchable circuit board and exhibiting electric properties changing according to the amount of moisture absorption, and a stretchable electrode provided on the main surface, having stretchability, and configured to measure the electric properties of the moisture absorbing film.

A moisture sensitive sheet <2> is the moisture sensitive sheet <1> which further includes an adhesive layer formed in a partial region of the stretchable circuit board and exhibiting adhesion.

A moisture sensitive sheet <3> is the moisture sensitive sheet <2> in which the adhesive layer is formed such that at least a part of the moisture absorbing film is exposed on the main surface provided with the moisture absorbing film.

A moisture sensitive sheet <4> is the moisture sensitive sheet <3> in which the stretchable circuit board includes a moisture impermeable film exhibiting low moisture permeability.

A moisture sensitive sheet <5> is the moisture sensitive sheet <2> in which the adhesive layer is formed such that at least a part of a surface on the back of a main surface region where the moisture permeable film is formed is exposed at the surface on the back of the main surface provided with the moisture absorbing film.

A moisture sensitive sheet <6> is the moisture sensitive sheet <5> in which the stretchable electrode includes multiple electrode portions, and the adhesive layer is formed such that at least a part of a surface on the back of a main surface region between the multiple electrode portions is exposed.

A moisture sensitive sheet <7> is the moisture sensitive sheet <5> or <6> in which the stretchable circuit board includes a moisture permeable film exhibiting moisture permeability.

A moisture sensitive sheet <8> is any one of the moisture sensitive sheets <5> to <7> which further includes a stretchable protection film covering the moisture absorbing film and having stretchability.

A moisture sensitive system <9> includes any one of the moisture sensitive sheets <1> to <8>, a property acquirer configured to acquire the electric properties measured by the stretchable electrode of the moisture sensitive sheet, a calculator configured to obtain a temporal change in the electric properties acquired by the property acquirer, and a signal output unit configured to output a signal based on a result of calculation of the calculator.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A moisture sensitive sheet comprising:
a stretchable circuit board having stretchability;
a moisture absorbing film provided on a main surface of the stretchable circuit board and exhibiting an electric property changing according to an amount of moisture absorption; and
a stretchable electrode provided on the main surface, having stretchability, and configured to measure the electric property of the moisture absorbing film.

2. The moisture sensitive sheet according to claim 1, further comprising:
an adhesive layer formed in a partial region of the stretchable circuit board and exhibiting adhesion.

3. The moisture sensitive sheet according to claim 2, wherein
the adhesive layer is formed such that at least a part of the moisture absorbing film is exposed on the main surface provided with the moisture absorbing film.

4. The moisture sensitive sheet according to claim 3, wherein
the stretchable circuit board includes a moisture impermeable film exhibiting low moisture permeability.

5. The moisture sensitive sheet according to claim 2, wherein
the adhesive layer is formed at a surface on a back of the main surface provided with the moisture absorbing film such that the surface on the back of the main surface corresponding to a first region is at least partially exposed, the moisture absorbing film being formed in the first region.

6. The moisture sensitive sheet according to claim 5, wherein
the stretchable electrode includes multiple electrode portions, and
the adhesive layer is formed such that the surface on the back of the main surface corresponding to a second region between the multiple electrode portions is at least partially exposed.

7. The moisture sensitive sheet according to claim 5, wherein
the stretchable circuit board includes a moisture permeable film exhibiting moisture permeability.

8. The moisture sensitive sheet according to claim 5, further comprising:
a stretchable protection film covering the moisture absorbing film and having stretchability.

9. A moisture sensitive system comprising:
the moisture sensitive sheet according to claim 1;
a property acquirer configured to acquire the electric property measured by the stretchable electrode of the moisture sensitive sheet;
a calculator configured to obtain a temporal change in the electric property acquired by the property acquirer; and
a signal output unit configured to output a signal based on a result of calculation of the calculator.
